(19) United States
(12) Patent Application Publication
(10) Pub. No.: US 2006/0029518 A1
(43) Pub. Date: Feb. 9, 2006

(54) Toothbrush sterilizer and cleaner

(76) Inventor: Mu-Yi Yu, Keelung City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/913,320

(22) Filed: Aug. 9, 2004

(51) Int. Cl. A61L 2/07

(52) U.S. Cl. 422/62; 422/300; 422/26

(57) ABSTRACT

A multiple purpose toothbrush sterilizer and cleaner including the tooth brush holder or the face cleaner hood or nose syringe hood, comprises a base for the steam chamber and a temperature control switch. The toothbrush holder, or the face cleaner hood or the nose syringe hood is inserted into the porthole of the casing body. The steam chamber is located under the porthole. The chamber lid has a rotary nozzle directing to the porthole. When the nozzle is taken off, the water can be refilled into the steam chamber. When the nozzle is turned in, the high temperature steam will gush out of the nozzle and direct to toothbrush, or the face or the nose for sterilizing and cleaning process. The condensed water will return to the steam chamber for reuse. The temperature control switch and the fuse work as the double protection while the heater continues heating but no water contained in the steam chamber.

### BACKGROUND OF THE INVENTION

1. Field of the Invention

This invention concerns multiple purpose toothbrush sterilizer and cleaner, in particular it is provided with an auto cut off and manual reset thermal switch to ensure that the steam generated in the steam chamber can be used as the toothbrush sterilizer and cleaner, face cleaner and nose syringe as well.

2. Description of the Related Art

After tooth brushing, there is much dirt left on the bristle of the toothbrush, which is usually hanged up in the wet bathroom without drying. After long time fermentation, bacteria are cultivated unhealthy to the user. Even there are available the toothbrush sterilizers in the market, but they present the following weak points for the user to apply:

1. The prior art of the steam sterilizer has set up the fixed time period for steam generation and sterilizing operation, the user has no way to know whether the water in the steam chamber is sufficient for continued operation. In case of low water level, continued heating would lead to overheating, shortening the service span and even an explosion or a fire.

2. The prior art of the steam sterilizer is uniquely designed for cleaning the toothbrush, no other application available. In most cases, women have to buy the face steamer to wipe off the dirt built up under the skin on/in their faces or to buy nose syringe to cure the nasal diseases. They are designed with a single purpose, not for exchangeable application. It is costly, not an excitement to stimulate the strong desire to purchase.

3. In operation of the prior art of steamer, the steam or the condensed water is easy to overflow or to filter into the interior of the steamer, hard for aftermath cleaning. The steamer, if not dried up, is easily susceptible to the generation of derivative pathogens.

It is apparent that the prior art of steamer exists many weak points, requiring prompt improvement.

The inventor knows well the weaknesses of the prior art of steamer and has devoted for years great efforts to improvement and finally come up with this multiple purpose toothbrush sterilizer and cleaner.

### SUMMARY OF THE INVENTION

The main object of this invention is to provide a heater with a set temperature, when the set temperature is reached, the switch will cut off the power automatically, and the user has to reset the switch if people wants to re-heat so as to ensure the operational safety of the multiple purpose tooth brush sterilizer and cleaner.

The 2nd object of this invention is to provide a fuse as the second protection. When the temperature control switch is out of work, the fuse will blow off to protect the multiple purpose tooth brush sterilizer and cleaner.

The 3rd object of this invention is to provide the steam a return overflow hole to return the condensed water back to the steam chamber for reuse so as to keep the working environment always dry and neat for the multiple purpose tooth brush sterilizer and cleaner.

The 4th object of this invention is that this toothbrush sterilizer and cleaner can be operated as the face cleaner and nose syringe as well.

To achieve these multiple purposes, the sterilizer and cleaner shall be outfitted with a toothbrush cleaner, a face cleaner hood and a nose syringe hood. The steam chamber has a base and a temperature control switch on which the base provides a same opening structure to receive the toothbrush cleaner box, the face cleaner hood and the nose syringe hood for exchange application. Under the opening and on the top lid of the steam chamber, there is an open channel to fit with a rotary nozzle. When the nozzle is turned off, the water can be refilled into the steam chamber. When the nozzle is turned in, it closes the open channel to the steam chamber. The high temperature steam generated in the steam chamber will gush out of the nozzle and spray into the toothbrush holder, face cleaner hood or the nose syringe hood. The condensed water in the hood will return to the steam chamber through the return overflow hole, not overflow outside of the steamer. The temperature control switch and the fuse work as a double protection when the water is consumed, the power is automatically cut off. This invention provides a multiple purpose, convenient, neat and safe steamer.

### BRIEF DESCRIPTION OF THE DRAWINGS

These drawings disclose an illustrative embodiment of the present invention, which serves to exemplify the various advantages and objects thereof, and are as follows:

FIG. 1 shows the disassembly of the multiple purpose toothbrush sterilizer and cleaner of this invention.

FIG. 2 shows the elevation of basic steamer of the multiple purpose toothbrush sterilizer and cleaner of this invention.

FIG. 3 shows the disassembly of the toothbrush holder of the multiple purpose toothbrush sterilizer and cleaner of this invention.

FIG. 4 shows the elevation of the toothbrush holder of the multiple purpose toothbrush sterilizer and cleaner of this invention.

FIGS. 5A and 5B show the steam is working on cleaning the toothbrush.

FIG. 6 shows another embodiment of the multiple purpose toothbrush sterilizer and cleaner of this invention.

FIG. 7 shows another embodiment of the multiple purpose toothbrush sterilizer and cleaner of this invention.

### DETAILED DESCRIPTION OF THE INVENTION

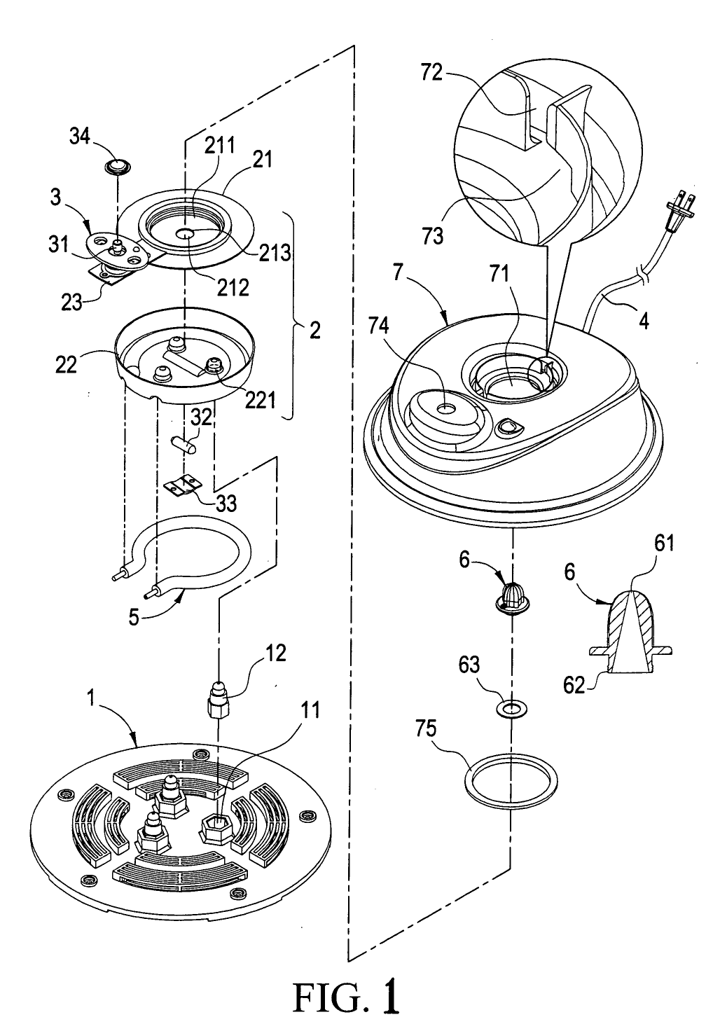

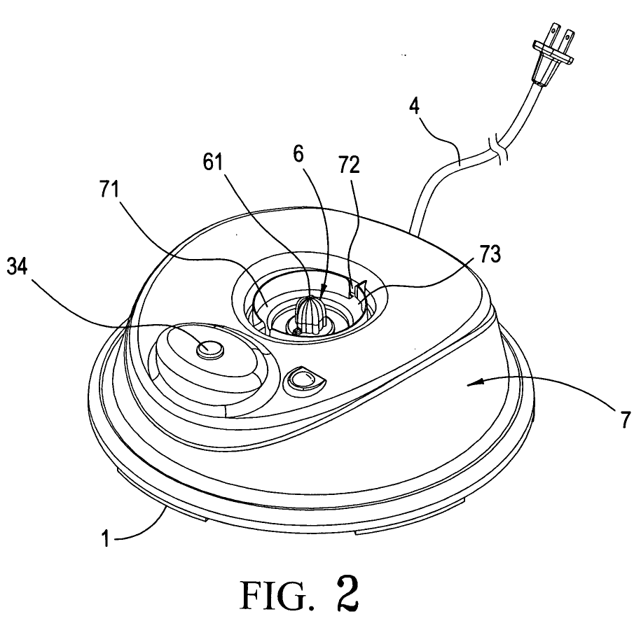

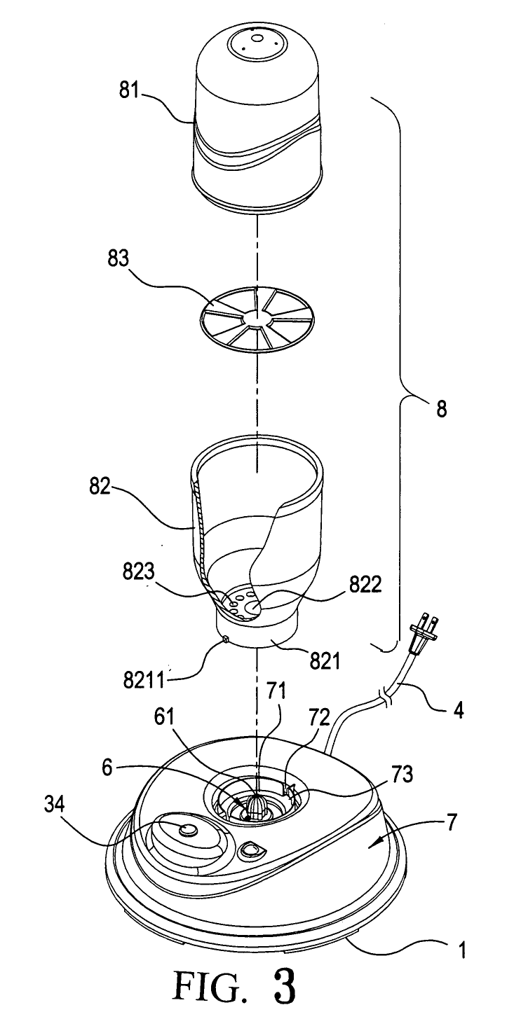

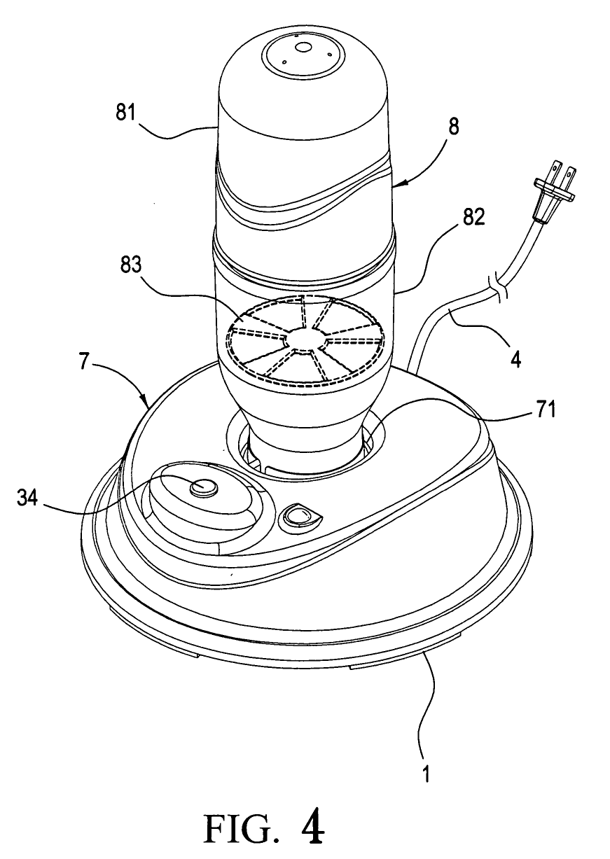

Please refer to FIGS. 1 through 4, the multiple purpose toothbrush sterilizer and cleaner of this invention mainly comprises:

A base 1 which has at least two retaining grooves 11 to receive the retaining posts 12 and each retaining post 12 has a projected top exposed outside the retaining groove 11.

A steam chamber 2 contains the chamber lid 21 and the chamber body 22. The chamber lid 21 has a recess 211 with a water refill hole 212 at the recess center. The wall of water refill hole 212 is cut with the inner threads 213. The chamber lid 21 has an extension board 23 to be mounted with the temperature control switch 3 and linked to the power cable 4. The temperature control switch 3 has a reset button 31. The chamber body 22 has at least two retaining grooves 221 on the bottom plate aligned with the retaining grooves 11 on the base 1. When the chamber lid 21 covers on the chamber body 22 to form the steam chamber 2, and the water is filled into the chamber body 22 through the water refill hole 212 on the chamber lid 21. The steam chamber 2 is installed on the top of the base 1 with the project tops 12 locked along the retaining grooves 221 in the chamber body 22, there keeps a gap between the base 1 and the steam chamber 2 in an effort to prevent the heat from transmitting to the base 1.

A heater 5 is in U form linked to the temperature control switch 3. The heater 5 is mounted between the base 1 and the chamber body 22 of the steam chamber 2 for heating the water in the steam chamber 2.

A fuse 32 is mounted in the cartridge board 33 under the bottom of the chamber body 22 of the steam chamber 2 and linked to the heater 5. In case the fuse is blown off, the power to the heater 5 is cut off.

A nozzle 6 has a gush mouth 61 on the tip. The interior of the nozzle 6 is in conical form in an attempt to gain great pressure on the steam gushing. The outer wall of the nozzle is cut with the outer threads 62 to be fitted with the inner threads 213 on the wall of the water refill hole 212 of the chamber lid 21. The nozzle 6 is located in the water refill hole 212 on the top of the chamber lid 21 and seals the water refill hole 212 therein. A waterproof ring 63 is provided between the nozzle 6 and the water refill hole 212 to ensure a leakage closure.

A casing body 7 has a joint porthole 71 and two join slots 72 along the porthole wall and a vertical buckle 73. An opening 74 is on the other side of the casing body 7. The casing body 7 sits on the base 1 with the porthole 71 aligned to the recess 211 of the chamber lid 21. The joint porthole 71 of the casing body 7 locks the chamber lid 21 and the chamber body 22 together with a sealing washer 75, which is in the joint between the joint porthole 71 and the chamber lid 21. The one side of the opening 74 on the casing 7 is for exposing the reset button 21 on the temperature control switch 3. The reset button 21 is provided with a waterproofing sleeve 34 to avert the filtration of water into the temperature switch 3.

A toothbrush holder 8 constitutes an upper case 81 and a lower case 82. A toothbrush rack 83 is provided in the toothbrush holder 8 to partition toothbrushes. The lower case 82 has a round bottom rim 821 and two projected catches 8211 on both sides. The lower case 82 has a central opening 822 with a plurality of holes 823 around the border. When the toothbrush holder 82 is inserted into the joint porthole 71, the project catch 821 on the lower case 82 will slide down along the joint slot 72 to be caught by the vertical buckle 73. When the toothbrush holder 8 is properly placed, the nozzle 6 will direct to the center opening 822 on the lower case 82.

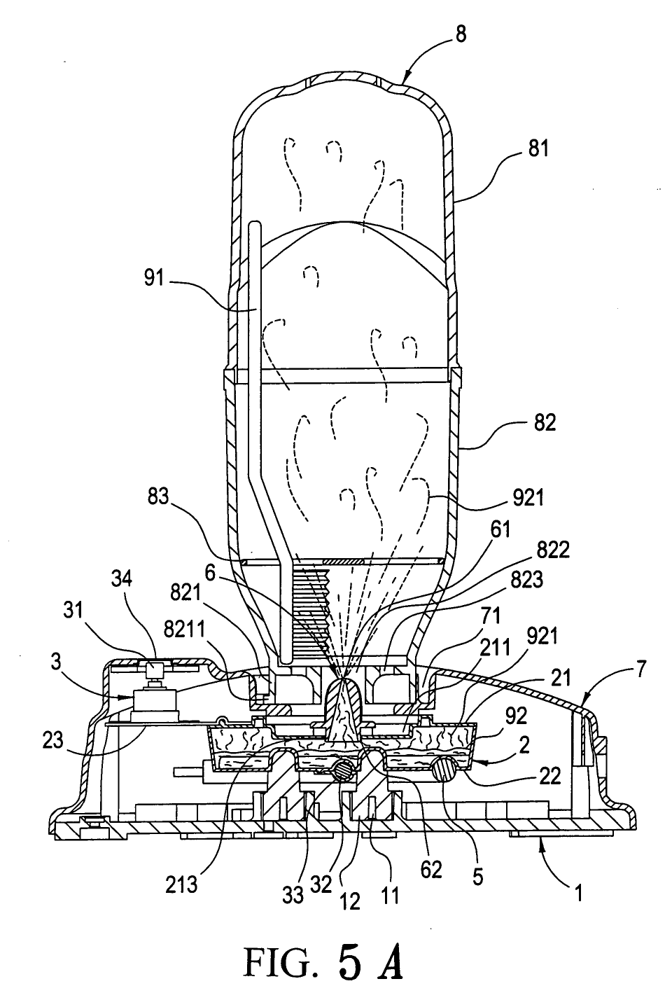

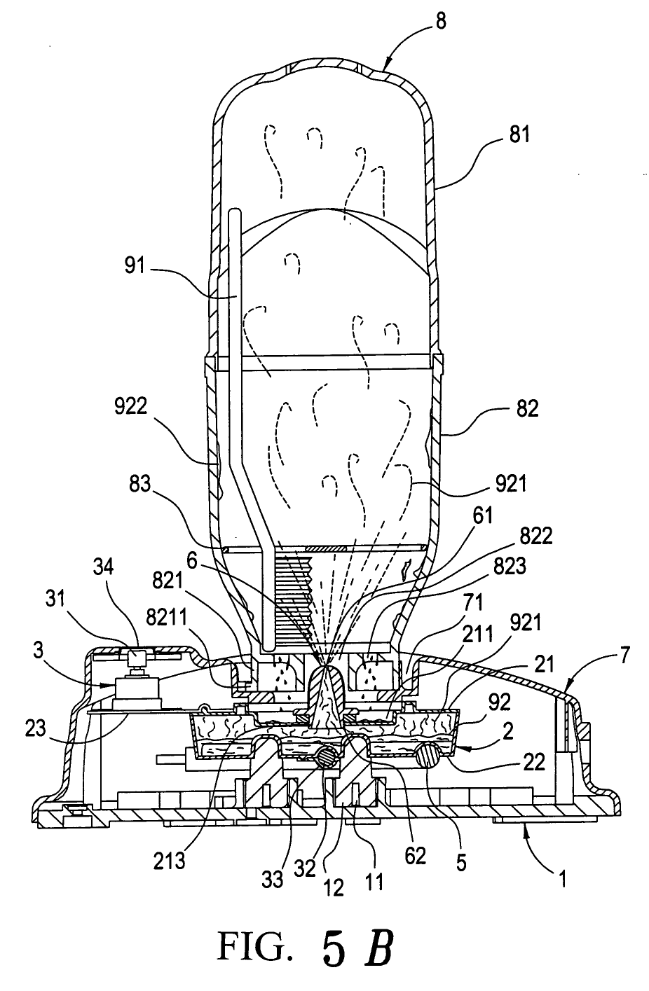

As shown in FIGS. 5A and 5B, during the course of sterilizing process, the toothbrush 91 is arranged along the rack 83 of the toothbrush holder 8 which is sitting in the joint porthole 71 of the casing body 7. When the heater 5 heats the steam chamber 2, the water 92 contained in the chamber body 22 will gradually become a steam 921 to gush out of the gushing mouth 61 of the nozzle 6 and direct to the toothbrushes for sterilizing and disinfecting. The used steam 921 will fall down to become the condensed water 922 returning to the steam chamber 2 by means of the water return hole 823 on the lower case 82 of the toothbrush holder 8 for reuse. When the temperature control switch 3, having detected that the heater 5 has reached the set temperature, will cut off power to the heater 5 to avoid any accident. So it requires a manual pressing the reset button 31 to re-start the heater 5. In case the temperature control switch 3 fails to work and the temperature in the steam chamber becomes abnormal, the fuse works as the second protection to the attainment of safety.

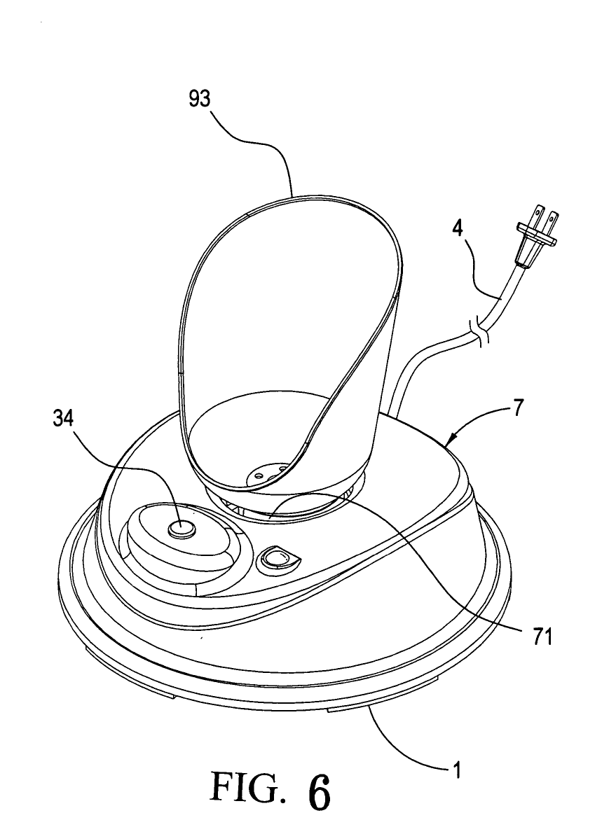

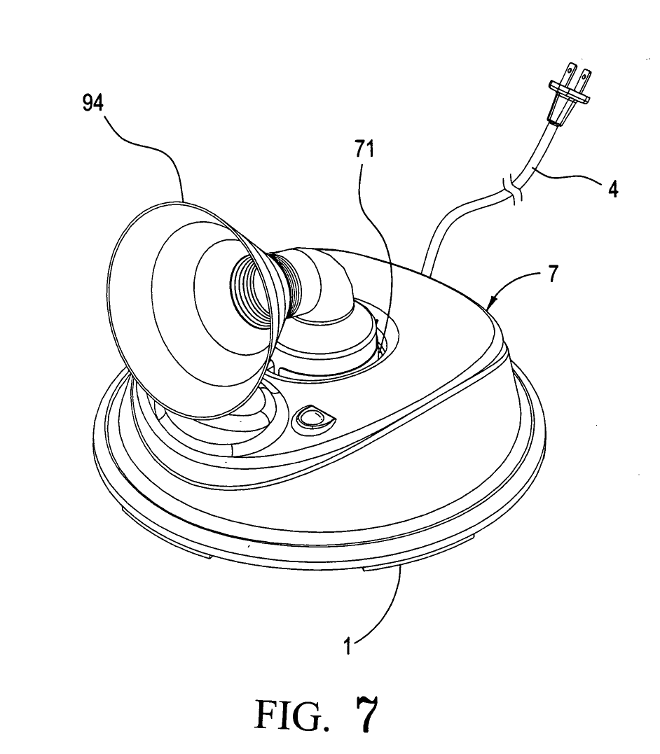

Apart from the sterilization and disinfection of toothbrushes, it can be used as the face cleaner 93 (FIG. 6) and the nose syringe 94 (FIG. 7) for user to replace the toothbrush holder 8 if necessary. The face cleaner hood 93 and the nose syringe hood 94 are designed to fit to the joint porthole 71 of the casing body 7, the steam generated 921 in the steam chamber 2 will come out of the nozzle 6 and gush over the face cleaner hood 93 or the nose syringe hood 94 to cleaning face or treating nose.

In case the user wants to refill the water, he has to take off the toothbrush holder 8 (or the face cleaner hood 93 or the nose syringe hood 94) first, at this moment, both the nozzle 6 and steam 921 are visible. If the steam 921 is still gushing, it reminds the user to shut off the power first. Turn off the nozzle 6 and refill the chamber body 22 with water through the water refills hole 212. Care shall be taken not to be inured with burn.

The multiple purpose toothbrush sterilizer and cleaner of this invention embraces the following advantages as compared with the prior art:

1. When the heating temperature reaches the set temperature, the temperature control switch will automatically cut off the power. If the user wants to re-start the heater, he has to press the reset button in an attempt to gain overall safety in operation.

2. This invention provides the fuse as the second protection in the event the temperature control switch fails to work.

3. The toothbrush holder provides a return flow hole, all condensed water will return to the steam chamber for reuse; the working environment is always kept in dry and neat condition.

4. Apart from the toothbrush sterilization and cleaning, it can be used as the face cleaner and the nose syringe.

The present invention is susceptible to variations and modifications, which may be introduced without departing from the inventive concept. It is to be understood that the present invention is not limited to the embodiments described above, but encompasses any and all embodiments within the scope of the following claims.

What the invention claimed is:

1. A multiple purpose tooth brush sterilizer and cleaner at least comprises:
A base having at least a retaining post;
A steam chamber having a chamber body and a chamber lid, the chamber lid has a water refill hole to be linked with a nozzle with the tip upward, the chamber lid has an extension board to be installed with the temperature control switch and reset button, the steam chamber is mounted on the retaining post of the base and a clearance is maintained between the steam chamber and the base to receive the heater which is controlled by the temperature control switch;
A fuse installed under the bottom of the steam chamber and connected to the heater;
A casing body having a joint porthole, a buckle and a connecting hole on one side, the joint porthole links to the base with the nozzle exposed outside, the reset button of the temperature control switch is mounted in the connecting hole;
A tooth brush holder consisting of the upper case and the lower case, the bottom of the lower case is locked in the buckle of the porthole of the casing body, the lower case has a center opening with a plurality of holes along its border, and the center opening is aligned to the nozzle tip;
When the heater reaches the set temperature, the temperature control switch will cut off the power; it requires manual resetting to restart the heater, and the fuse provides the second protection to the heater.

2. The multiple purpose toothbrush sterilizer and cleaner as claimed in the claim 1 in which the toothbrush holder has rack to separate the toothbrushes inside.

3. The multiple purpose tooth brush sterilizer and cleaner as claimed in the claim 1 in which the reset button of the temperature control switch is outfitted with water proofing sleeve to prevent the water from filtering and causing the temperature control switch ineffective.
